(19) United States
(12) Patent Application Publication
(10) Pub. No.: US 2006/0115445 A1
(43) Pub. Date: Jun. 1, 2006

(54) Compositions for treating keratin materials comprising electrophilic monomers and liquid crystal agents

(76) Inventors: Gaelle Brun, Paris (FR); Luc Gourlaouen, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,332

(22) Filed: Oct. 13, 2005

Related U.S. Application Data
(60) Provisional application No. 60/637,750, filed on Dec. 22, 2004.

(30) Foreign Application Priority Data
Oct. 13, 2004 (FR) 04 10812

(51) Int. Cl. A61K 8/81

(52) U.S. Cl. 424/70.16

(57) ABSTRACT

Disclosed herein is a cosmetic composition for treating keratin materials, for example, keratin fibers such as the hair, comprising, in a cosmetically acceptable medium, at least one electrophilic monomer and at least one liquid crystal agent. Also disclosed is a method for cosmetically treating keratin materials comprising applying the cosmetic composition to the keratin materials.

This application claims benefit of U.S. Provisional Application No. 60/637,750, =filed Dec. 22, 2004, the contents of which are incorporated herein by reference. This application also claims benefit of priority under 35 U.S.C. § 119 to French Patent Application No. 04 10812, filed Oct. 13, 2004, the contents of which are also incorporated by reference.

The present disclosure relates to compositions for treating keratin materials, for instance keratin fibers such as the hair, comprising in situ polymerizable monomers, a cosmetically acceptable medium, and liquid crystal agents, the use thereof, and methods for treating keratin materials. This disclosure also relates to compositions based on electrophilic monomers and liquid crystal agents.

To enable a hair color to be visible against a dark background, without the coloration being a simple glint, it is necessary to lighten the keratin materials. Several methods are known to achieve his goal. For example, in commercially available products, keratin materials, such as hair, are lightened by bleaching the melanin of the hair with an oxidant. However, this process harms the hair. Further, keratin fibers may also be lightened by depositing a pigment on the surface of the fiber. Lightening occurs because the pigment masks the natural color of the fiber, and thus is visible even against a dark background. However, the deposits that result from this process do not withstand shampooing.

A relatively limited range of colorants is currently employed to color keratin materials. In particular, lake, mineral, or nacreous pigments are utilized. The lakes allow vivid colors to be obtained, but are unstable to light, temperature and pH, and may stain the scalp if applied to the hair. In contrast, mineral pigments, such as mineral oxides are highly stable, but impart relatively dull, pale colors to keratin fibers. Finally, nacreous pigments may be used to impart a variety of colors to keratin fibers. However, these colors, though often iridescent, are not intense and are usually weak.

The Applicant has discovered, surprisingly, that it is possible to obtain new coloring effects by employing a mixture of electrophilic monomers, such as those described in French Patent Application No. FR 2840208, a cosmetically acceptable medium, and liquid crystal agents. When this composition is applied to keratin material, such as hair, colors are obtained that vary with the incident angle of the light and the angle of observation. Hair that is treated with this composition is conditioned and exhibits good sheen. Individual hairs that have been treated with this composition remain perfectly separate and can be styled without problem. Further, this composition forms a coating on keratin fibers that withstands several shampooings. The conditioning and the sheen of fiber also withstand shampooing.

One aspect of the present disclosure is a composition for treating keratin materials, such as the hair, comprising, in a cosmetically acceptable medium, at least one electrophilic monomer and at least one liquid crystal agent. A further aspect of the present disclosure is a method for treating keratin fibers with the aforementioned composition. Yet another aspect of the present disclosure is a kit comprising a first composition containing at least one electrophilic monomer and optionally at least one free-radical and/or anionic polymerization inhibitor, and a second composition comprising in a cosmetically acceptable medium at least one liquid crystal agent.

Other non-limiting subjects, characteristics, aspects and advantages of the disclosure will become evident to one of ordinary skill upon reading the description and the various examples that follow.

For the purpose of the present disclosure, the term, “electrophilic monomer” refers to those monomers that are monomer capable of undergoing polymerization by anionic polymerization in the presence of a nucleophile, e.g. the hydroxyl (OH—) ions present in water. The term “anionic polymerization” refers to the mechanism disclosed in “Advanced Organic Chemistry”, Third Edition, Jerry March, pages 151 to 161. The term “electron-withdrawing group” or “inductively withdrawing group (—I)” as used herein, refers to any group that is more electronegative than carbon. Reference may be made to the work by P. R. Wells in Prog. Phys. Org. Chem., Vol. 6, 111 (1968). Conversely, a “minimally or non-electron-withdrawing group” refers to any group whose electronegativity is less than or equal to that of carbon.

Examples of the at least one electrophilic monomer utilized in the present disclosure include, but are not limited to:

monomers of formulas A through B1 below,

benzylidenemalononitrile derivatives (A), 2-(4-chlorobenzylidene)malononitrile (A1), ethyl 2-cyano-3-phenylacrylate (B) and ethyl 2-cyano-3-(4-chlorophenyl)acrylate (B1), as described in Sayyah, J. Polymer Research, 2000, p. 97

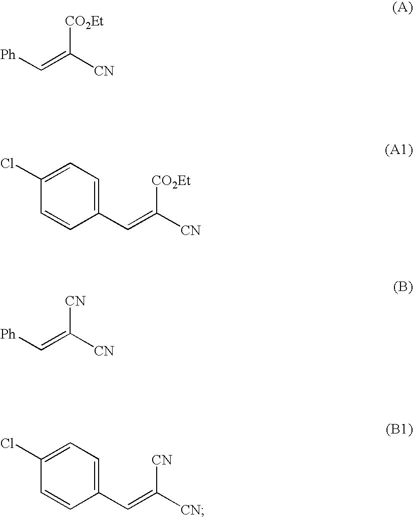

methylidenemalonate derivatives such as diethyl 2-methylenemalonate (C), as described by Hopff, Makromolekulare Chemie, 1961, p. 95, by De Keyser, J. Pharm. Sci, 1991, p. 67, and by Klemarczyk, Polymer, 1998, p. 173

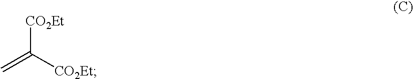

ethyl 2-ethoxycarbonylmethyleneoxycarbonylacrylate (D), as described by Breton, Biomaterials, 1998, p. 271 and Couvreur, Pharmaceutical Research, 1994, p. 1270:

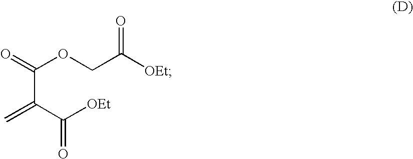

itaconate derivatives, such as dimethyl itaconate (E), as described by Bachrach, European Polymer Journal, 1976, p. 563

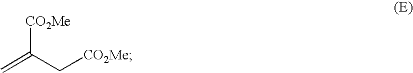

itaconimide derivatives, such as N-butylitaconimide (F), N-(4-tolyl)itaconimide (G), N-(2-ethylphenyl)itaconimide (H) and N-(2,6-diethylphenyl)itaconimide (I), as described by Wanatabe, J. Polymer Science: Part A: Polymer Chemistry, 1994, p. 2073 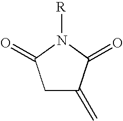
R═Bu (F), 4-tolyl (G), 2-ethylphenyl (H), 2,6-diethylphenyl (I)

methyl α-(methylsulphonyl)acrylate derivatives (K), ethyl α-(methylsulphonyl)acrylate derivatives (L), methyl α-(tert-butylsulphonyl)acrylate derivatives (M), tert-butyl α-(methylsulphonyl)acrylate derivatives (N) and tert-butyl α-(tert-butylsulphonyl)acrylate derivatives (O), as described by Gipstein, J. Org. Chem, 1980, p. 1486, and

1,1-bis(methylsulphonyl)ethylene derivatives (P), 1-acetyl-1-methylsulphonylethylene derivatives (O), methyl α-(methylsulphonyl)vinylsulphonate derivatives (R) and α-methylsulphonylacrylonitrile derivatives (S), as described by U.S. Pat. No. 2,748,050 to Shearer:

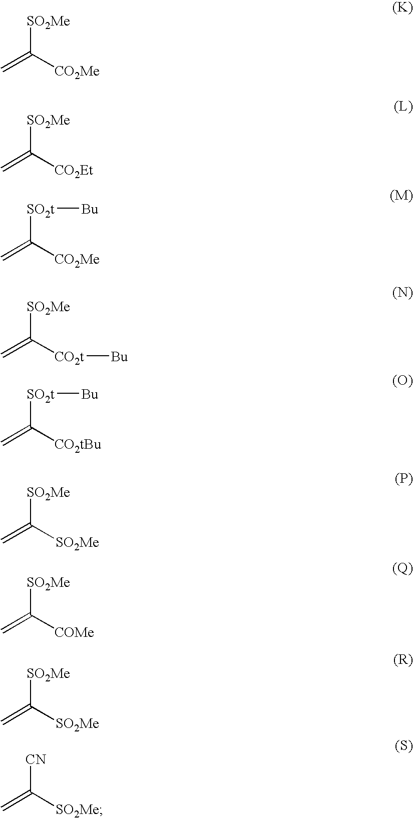

methyl vinyl sulphone derivatives (T) and phenyl vinyl sulphone derivatives (U), as are described by Boor, J. Polymer Science, 1971, p. 249

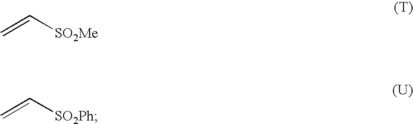

the phenyl vinyl sulphoxide derivative (V), as described by Kanga, Polymer Preprints (ACS, Division of Polymer Chemistry), 1987, p. 322

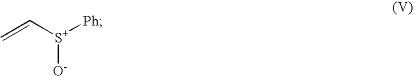

the 3-methyl-N-(phenylsulphonyl)-1-aza-1,3-butadiene derivative (W), as described by Bonner, Polymer Bulletin, 1992, p. 517

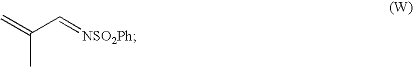

acrylate and acrylamide derivatives, such as N-propyl-N-(3-triisopropoxysilylpropyl)acrylamide (X) and N-propyl-N-(3-triethoxysilylpropyl)acrylamide (Y), as described by Kobayashi, Journal of Polymer Science, Part A: Polymer Chemistry, 2005, p. 2754

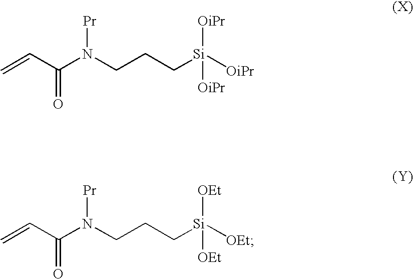

2-hydroxyethyl acrylate (Z) and 2-hydroxyethyl methacrylate (AA), as are described by Rozenberg, International Journal of Plastics Technology, 2003, p. 17

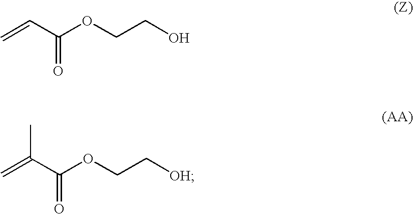

n-butyl acrylate (AB), as described by Schmitt, Macromolecules, 2001, p. 2115, and tert-butyl acrylate (AC), as described by Ishizone, Macromolecules, 1999, p. 955

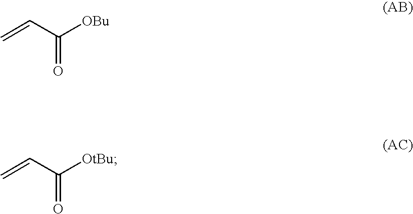

Electrophilic monomers that are suitable for use in the present invention may be cyclic or linear. In a non-limiting embodiment, when the electron-withdrawing monomer is cyclic, the electron-withdrawing group may be exocyclic, e.g., it does not form an integral part of the cyclic structure of the monomer).

In a further non-limiting embodiment of the present disclosure, the electron withdrawing monomers have at least two electron-withdrawing groups. Non-limiting examples of monomers having at least two electron-withdrawing groups include the monomers of formula (I) below, which may be obtained by free-radical polymerization, by polycondensation or by ring opening:

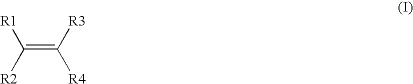

wherein:

R₁ and R₂, which may be the same or different, are chosen from, a minimally or non-electron-withdrawing group (a group with little or no inductive withdrawal effect), such as:
a hydrogen atom;
a saturated or unsaturated linear, branched or cyclic hydrocarbon group that optionally contains one or more nitrogen, oxygen and/or sulphur atoms, is optionally substituted by one or more groups chosen from —OR, —COOR, —COR, —SH, —SR, —OH and halogen atoms, and contains from 1 to 20 carbon atoms, for example, from 1 to 10 carbon atoms;
a modified or non-modified polyorganosiloxane residue, and;
a polyoxyalkylene group;

R₃ and R₄, which may be identical or different, each denote an electron-withdrawing (inductively withdrawing) group, including, but not limited to, —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, —OH, linear or branched alkenyl groups, linear or branched alkynyl groups, C₁-C₄ mono- or polyfluoroalkyl groups, aryl groups such as phenyl, or aryloxy groups such as phenyloxy; and

R is chosen from saturated, unsaturated, linear, branched and cyclic hydrocarbon group that contains from 1 to 20 carbon atoms, for example, from 1 to 10 carbon atoms, that optionally contains one or more nitrogen, oxygen and/or sulphur atoms, and is optionally substituted by one or more groups chosen from —OR′, —COOR′, —COR′, —SH, —SR′, —OH and halogen atoms, and a polymer residue that is obtainable by free radical polymerization, by polycondensation, or by ring opening, in which R′ denotes a C₁-C₁₀ alkyl radical.

In a non-limiting embodiment of the present disclosure, the alkenyl or alkynyl groups that may be utilized in formula (I) contain from 2 to 20 carbon atoms, for example, from 2 to 10 carbon atoms.

Examples of the saturated or unsaturated linear, branched or cyclic hydrocarbon group that may be utilized as R of formula (I) include, but are not limited to linear or branched alkyl, alkenyl or alkynyl groups, such as methyl, ethyl, n-butyl, tert-butyl, isobutyl, pentyl, hexyl, octyl, butenyl or butynyl; and cycloalkyl or aromatic groups.

Non-limiting examples of substituted hydrocarbon groups that may be utilized in formula (I) include hydroxyalkyl or polyhaloalkyl groups.

Non-limiting examples of non-modified polyorganosiloxanes that may be utilized in formula (I) include polyalkylsiloxanes, such as polydimethylsiloxanes, polyarylsiloxanes, such as polyphenylsiloxanes, and polyarylalkylsiloxanes, such as polymethylphenylsiloxanes.

Examples of modified polyorganosiloxanes that may be utilized in formula (I) include, but are not limited to polydimethylsiloxanes containing one or more polyoxyalkylene, siloxy, silanol, amine, imine, or fluoroalkyl groups.

Non-limiting examples of polyoxyalkylene groups that may be utilized in formula (I) include polyoxyethylene groups and polyoxypropylene groups, including those having from 1 to 200 oxyalkylene units.

Non-limiting examples of mono- or polyfluoroalkyl groups that may be utilized in formula (I) include —(CH₂)ₙ—(CF₂)ₘ—CF₃ or —(CH₂)ₙ—(CF₂)ₘ—CHF₂, where n=1 to 20 and m=1 to 20.

The substituents R₁ to R₄ may optionally be substituted by a group that has cosmetic activity. In a non-limiting embodiment of the present disclosure, groups having coloring, antioxidant, UV filter and conditioning functions are utilized.

Examples of groups having a coloring function include, but are not limited to, azo, quinone, methine, cyanomethine and triarylmethane groups.

Non-limiting examples of groups having an antioxidant function include groups of the butylated hydroxyanisole (BHA) type, butylated hydroxytoluene (BHT) type or vitamin E type.

Examples of groups having a UV filter function include, but are not limited to groups of the benzophenone, cinnamate, benzoate, benzylidenecamphor and dibenzoylmethane types.

Non-limiting examples of groups having a conditioning function include cationic groups and fatty acid ester type groups.

In a further non-limiting embodiment, the electrophilic monomer is chosen from cyanoacrylates and cyanoacrylate derivatives, of formula (II):

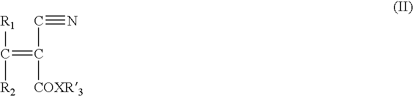

wherein:

X is chosen from NH, S, and O; R₁ and R₂ have the same significations as disclosed for formula (I) above,

R′₃ denotes a hydrogen atom or a radical R as defined for formula (I) above.

In one non-limiting embodiment, X is O.

Non-limiting examples of electrophilic monomers of formula (II) include:

a) C₁₋₂₀ polyfluoroalkyl 2-cyanoacrylate monomers, such as the 2,2,3,3-tetrafluoropropyl ester of 2-cyano-2-propenoic acid, of the formula: 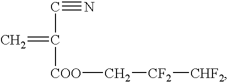
or the 2,2,2-trifluoroethyl ester of 2-cyano-2-propenoic acid, of the formula:

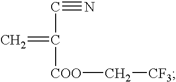

and

b) C₁-C₁₀ alkyl or (C₁-C₄ alkoxy)(C₁-C₁₀ alkyl) cyanoacrylates.

Non-limiting examples of monomers a) and b) that may be utilized in the present disclosure include ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyano-acrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

In a non-limiting embodiment of the present disclosure, monomer b) is utilized as the electrophilic monomer.

In another non-limiting embodiment of the present disclosure, the electrophilic monomer is chosen from monomers of formula (III) below, and mixtures thereof:

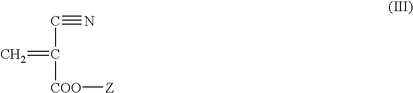

wherein, Z is chosen from (CH₂)₇—CH₃, CH(CH₃)—(CH₂)₅—CH₃, CH₂—CH(C₂H₅)—(CH₂)₃—CH₃, (CH₂)₅—CH(CH₃)—CH₃, and (CH₂)₄—CH(C₂H₅)—CH₃.

The monomers used in accordance with the invention may be attached covalently to supports such as polymers, oligomers or dendrimers. Non-limiting examples of polymer or oligomer supports that may utilized in the present disclosure include, but are not limited to those having a linear, branched, comb or block structure. The distribution of the monomers of the disclosure over the polymeric, oligomeric or dendritic structure may be random, terminal or blockwise.

As used herein, the term, “cosmetically acceptable medium” refers to a medium that is compatible with keratin materials, such as the hair.

The cosmetically acceptable medium may be anyhydrous. As used herein, the term, “anhydrous medium” refers to a medium that contains less than 1% by weight of water relative to the total weight of the composition.

In a non-limiting embodiment of the present disclosure, the cosmetically acceptable medium is selected from organic oils; silicones such as volatile silicones, amino or non-amino silicone gums or oils and mixtures thereof; mineral oils; vegetable oils such as olive oil, castor oil, colza oil, copra oil, wheatgerm oil, sweet almond oil, avocado oil, macadamia oil, apricot oil, safflower oil, candlenut oil, false flax oil, tamanu oil and lemon oil; waxes; or organic compounds such as C₅-C₁₀ alkanes, acetone, methyl ethyl ketone, esters of C₁-C₂₀ acids and C₁-C₈ alcohols such as methyl acetate, butyl acetate, ethyl acetate and isopropyl myristate, dimethoxyethane, diethoxyethane; C₁₀-C₃₀ fatty alcohols such as lauryl alcohol, cetyl alcohol, stearyl alcohol and behenyl alcohol, C₁₀-C₃₀ fatty acids such as lauric acid and stearic acid, C₁₀-C₃₀ fatty amides such as lauric diethanolamide, C₁₀-C₃₀ fatty alcohol esters such as C₁₀-C₃₀ fatty alcohol benzoates, and mixtures thereof.

In a non-limiting embodiment of the present disclosure, the organic compounds are chosen from compounds that are liquid at a temperature of 25° C. and under a pressure of 10⁵ Pa (760 mmHg).

The composition of the present disclosure may contain the electrophilic monomer in a concentration ranging from 0.001% to 80% by weight, such as from 0.1 to 40% by weight, for example, from 1% to 20% by weight, relative to the total weight of the composition.

The composition of the present disclosure may further comprise polymerization inhibitors, such as free-radical and/or anionic polymerization inhibitors in order to increase the stability of the composition over time. Non-limiting examples of polymerization inhibitors that may be utilized in the present disclosure include sulphur dioxide, nitric oxide, lactone, boron trifluoride, hydroquinone and its derivatives such as hydroquinone monoethyl ether, tert-butylhydroquinone (TBHQ), benzoquinone and its derivatives such as duroquinone, catechol and its derivatives such as tert-butylcatechol and methoxycatechol, anisole and its derivatives such as methoxyanisole, hydroxyanisole or butylated hydroxyanisole, pyrogallol, 2,4-dinitrophenol, 2,4,6-trihydroxybenzene, p-methoxyphenol, hydroxybutyltoluene, alkyl sulphates, alkyl sulphites, alkyl sulphones, alkyl sulphoxides, alkyl sulphides, mercaptans, 3-sulpholene, and mixtures thereof. In a further non-limiting embodiment, the alkyl groups of the polymerization inhibitor are groups having 1 to 6 carbon atoms.

Organic or inorganic acids may also be utilized in the present composition as a polymerization inhibitor. Non-limiting examples of inorganic acids that may be utilized include inorganic acids having one or more carboxylic or sulphonic groups and which have a pKa of between 0 and 6, such as phosphoric acid, hydrochloric acid, nitric acid, benzene- or toluene-sulphonic acid, sulphuric acid, carbonic acid, hydrofluoric acid, acetic acid, formic acid, propionic acid, benzoic acid, mono-, di- or trichloroacetic acids, salicylic acid and trifluoroacetic acid.

The composition of the present disclosure may contain the polymerization inhibitor in an amount ranging from 10 ppm to 20%, such as from 10 ppm to 5%, for example, from 10 ppm to 1% by weight, relative to the total weight of the composition.

The compositions in accordance with the disclosure may further comprise one or more additives, including, but not limited to agents commonly used in cosmetology, such as reducing agents, fats, plasticizers, softeners, antifoams, moisturizers, pigments, clays, mineral fillers, UV filters, mineral colloids, peptizers, solubilizers, perfumes, preservatives, anionic, cationic, nonionic or amphoteric surfactants, fixative or non-fixative polymers, polyols, proteins, vitamins, direct dyes or oxidation dyes, pearlizers, propellants, and organic or inorganic thickeners such as benzylidenesorbitol and N-acylamino acids. These agents may be encapsulated, for example, within a polycyanoacrylate or encapsulating material.

As used herein, the term, “liquid crystal agent” refers to compounds that generate a mesomorphic state. That is, this term refers to materials that generate a state for which the melting of the crystals results in a liquid that possess optical properties comparable with those of certain crystals. These compounds are discussed in the liquid crystals chapter of the Ullmann encyclopaedia.

Liquid crystal agents are also discussed in the following patents or patent applications: EP 545 409; WO 94109086; EP 709 445; GB 2 282 145; GB 2 276 883; WO 95132247; WO 95132248; EP 686 674; and EP 711 780.

Non-limiting examples of compounds generating a mesomorphic state that may be utilized in the present disclosure include:

compounds having a cholesteric function, whose structure is as follows:

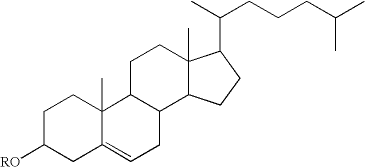

wherein:

R is chosen from alkyl and alkylcarbonyl groups containing from 1 to 30 carbon atoms which may be branched or unbranched, unsubstituted or substituted by cyclic, aromatic and/or halogen groups.

Non-limiting examples of liquid crystal agents having a cholestric function as discussed above include:

cholesterol erucyl carbonate; cholesterol methyl carbonate, cholesterol oleyl carbonate, cholesterol para-nonylphenyl carbonate, cholesterol phenyl carbonate, cholesterol acetate, cholesterol benzoate, cholesterol butyrate, cholesterol isobutyrate, cholesterol chloride, cholesterol chloroacetate, cholesterol cinnamate, cholesterol crotanoate, cholesterol decanoate, cholesterol erucate, cholesterol heptanoate, cholesterol hexanoate, cholesterol myristate, cholesterol nonanoate, cholesterol octanoate, cholesterol oleate, cholesterol propionate, cholesterol valerate and dicholesteryl carbonate; and compounds defined by the following formula:
Aₗ^{5′}-[(X¹)ₐ-(A¹)_{b}-(A²)_{c}]_{d}-Zₑ-[(X²)_{f}-(A³)_{g}-(A⁴)ₕ]ᵢ-Aₖ⁵

wherein,

X¹ and X², which may be the same or different, are chosen from divalent —O—, —COO—, —CONH—, —CO—, —S—, —C≡C—, —CH═CH—, —CH₂—CH₂, —CH═N—, —N═N—, —N═N(O)—, —CH═N—N═CH—, —CH═CH—COO— and —OCO—CH═CH— radicals,

A¹, A², A³ and A⁴, which may be the same or different, are chosen from divalent 1,4-phenylene, 1,4-cyclohexylene and optionally substituted arylene, heteroarylene, heterocycloalkylene and cycloalkylene radicals,

Z represents, is chosen from divalent to tetravalent, benzene-1,4-cyclohexane and benzene-1,3-cyclopentane radicals,

A⁵ and A^{5′}, which may be the same or different, are chosen from saturated, unsaturated alkyl, alkoxy and cycloalkyl radicals having 1 to 16 carbon atoms, a steroidal radical, a halogen, a hydrogen atom, hydroxyl, nitrile and trialkylsilyloxy radicals,

a, b, c, d, f, g, h, i, k, and l independently represent an integer ranging from 0 to 3,

e is 0 or 1,

the sum a+b+c+d+e+f+g+h+i+k being greater than or equal to 2, and the sum d+i being less than or equal to 4, with the proviso that this group does not include a peroxide radical.

Non-limiting examples of liquid crystal agents conforming to the formula:
Aₗ^{5′}-[(X¹)ₐ-(A¹)_{b}-(A²)_{c}]_{d}-Zₑ-[(X²)_{f}-(A³)_{g}-(A⁴)ₕ]ᵢ-Aₖ⁵ (I)

include, but are not limited to:

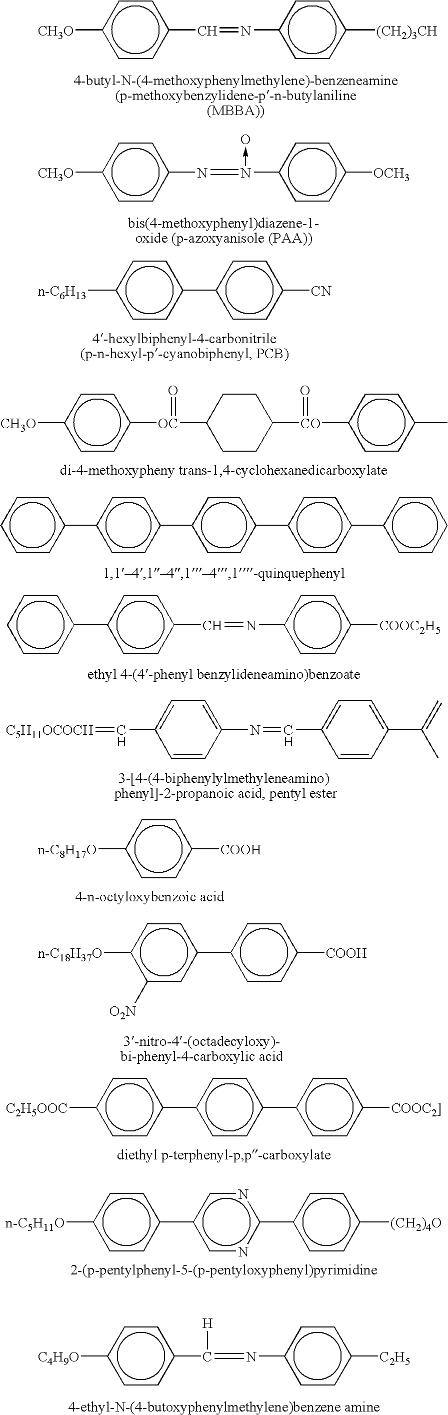

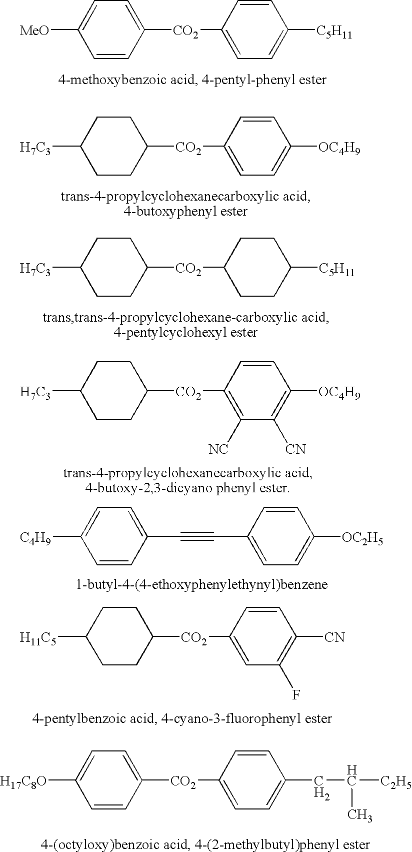

and polymers obtained by the polymerization of mesogenic monomers, such as those described in EP 1302524, EP 1304161, EP 1422283, WO 02/086609 and GB 2337753.

As used herein, the term, “mesogenic monomer” refers to a monomer that gives liquid crystal properties to the polymers obtained from these monomers (as defined in IUPAC, 2^{nd} edition, 1997). Mesogenic monomers have the general structure:
Y¹—B¹-M-R

in which:

Y¹ represents a polymerizable group chosen from: acrylate, methacrylate, epoxy, isocyanate, hydroxyl, vinyl ether (—O—CH═CH₂), vinyl ester (—CO—O—C═CH₂), styryl and trialkoxysiloxy groups;

B¹ represents a —CₙH₂ₙ— group, where n is an integer ranging from 0 to 20 and one or more methylene groups of the —CₙH₂ₙ— group may be substituted by a halogen atom or may be replaced (when the methylenes are not adjacent) by one or more groups chosen from: —O—, —NH—, —OCO—, —OCO—O—, —S—CO—COO—, —CONH—, —CO—, —S—, —C≡C—, and —CH═CH—,

M represents a group of formula:
—[(X¹)ₐ-(A¹)_{b}-(A²)_{c}]_{d}-Zₑ-[(X²)_{f}-(A³)_{g}-(A⁴)ₕ]ᵢ-

in which:

X¹ and X², which may be the same or different, are chosen from divalent —O—, —COO—, —CONH—, —CO—, —S—, —C≡C—, —CH═CH—, —CH₂—CH₂—, —CH═N—, —N═N—, —N═N(O)—, —CH═N—N═CH—, —CH═CH—COO— and —OCO—CH═CH— radicals;

A¹, A², A³ and A⁴, which may be the same or different, are chosen from divalent 1,4-phenylene, 1,4-cyclohexylene, optionally substituted arylene, heteroarylene and cycloalkylene, and heterocycloalkylene radicals;

Z is chosen from divalent to tetravalent, benzene-1,4-cyclohexane, benzene-1,3-cyclopentane radicals, and a divalent chiral group containing at least 4 carbon atoms, such as 4 to 20 carbon atoms, for example, 4 to 10 carbon atoms. In a non-limiting embodiment, the divalent chiral group contains at least one asymmetric carbon. In a further non-limiting embodiment, the divalent chiral group contains 2 asymmetric carbons.

In a further non-limiting embodiment, the chiral group originates from a group chosen from dianhydrohexatols, hexoses, pentoses, binaphthyl derivatives (binaphthyl groups), biphenyl derivatives (biphenyl groups), tartaric acid derivatives or optically active glycols. Non-limiting examples of chiral groups that may be utilized in the present disclosure include those described in WO 98/00428.

In another non-limiting embodiment of the present disclosure, when Z is chiral, it denotes a chiral group having two bonds, derived from the dianhydrohexitol group, such as a radical of formula:

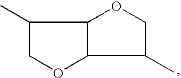

a, b, c, f, g, and h, which may be the same or different, represent an integer ranging from 0 to 3,

e is 0 or 1,

the sum a+b+c+d+e+f+g+h+i+k is greater than or equal to 2,

R is chosen from saturated and unsaturated alkyl, alkoxy and cycloalkyl radicals having 1 to 16 carbon atoms, a steroidal radical, a halogen, a hydrogen atom, a hydroxyl, nitrile and trialkylsilyloxy radicals, and a cholesteryl radical. R may also represent a structure of type B²—Y², wherein the definitions of B² and Y² are identical to the definitions of B¹ and Y¹ above. Y¹ and Y², and B¹ and B2 may be identical or different.

Examples of mesogenic monomers of the formula Y¹—B¹-M-R include, but are not limited to:

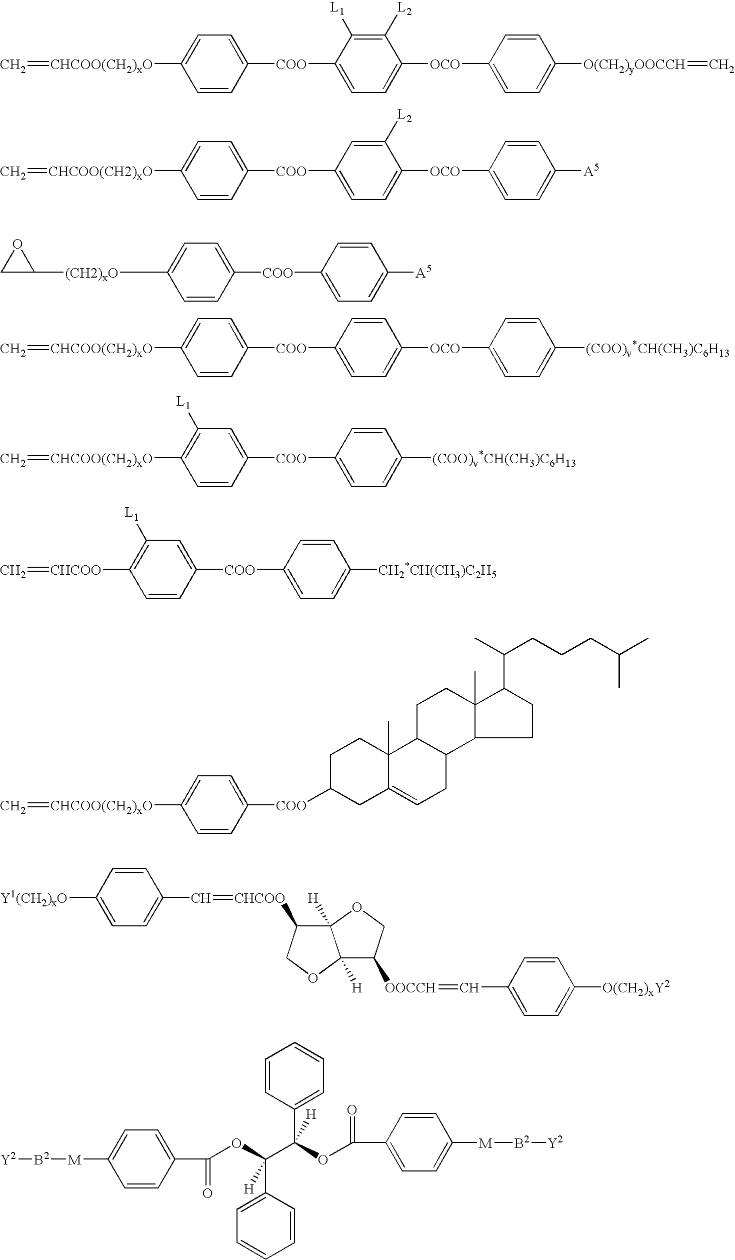

wherein,

L₁ and L₂, which may be identical or different, are chosen from H, F, Cl, CN, alkyl, alkoxy, alkylcarbonyl, halogenated alkyl, alkoxycarbonyl and alkoxycarbonyloxy groups having from 1 to 7 carbon atoms; and

x is an integer ranging from 0 to 20.

In a non-limiting embodiment of the disclosed composition, the liquid crystal agent is different from cholesteric liquid crystal agents.

In a further non-limiting embodiment of the present disclosure, the liquid crystal polymer may be obtained from the polymerization of a mesogenic monomer alone or together with one or more other mesogenic or non-mesogenic monomers. For example, the liquid crystal polymer may be obtained from the polymerization of one or more mesogenic monomers. In one non-limiting embodiment, the liquid-crytal polymer is obtained by polymerizing at least one polymerizable mesogenic compound having one polymerizable functional group and at least one polymerizable mesogenic compound having two or more polymerizable functional groups.

In another non-limiting embodiment of the present disclosure, the polymerization is carried out only with polymerizable mesogenic compounds having two or more polymerizable functional groups to form a polymer network.

In another non-limiting embodiment of the present invention, the final polymer is obtained by the polymerization of a chiral polymerizable mesogenic monomer and a non-chiral polymerizable mesogenic monomer.

Non-limiting examples of methods by which polymerizable mesogenic compounds containing mono-, di- or multi-reactive functions may be prepared according are described, for example, in Houben-Weyl, Methoden der organischen Chemie, Thieme Verlag, Stuttgart. Other non-limiting examples are described in patents WO 93/22397, EP 0261712, DE 19504224, DE 4408171, DE 4405316, U.S. Pat. No. 5,362,315, and U.S. Pat. No. 5,807,497.

The conditions for polymerizing the above monomers are those conventionally used for the reactive functions carried by the monomers. A non-limiting example of the mode of polymerization is described in patent EP 1302524.

In a non-limiting embodiment of the present disclosure, the liquid crystal agent is one of the polymers disclosed in patent application EP 1046692. Non-limiting examples of liquid crystal polymer particles conforming to the above definition include those known under the CTFA name Polyacrylate-4 and sold under the names Helicone® HC Sapphire, Helicone® HC Scarabeus, Helicone® HC Jade, Helicone® HC Maple, Helicone® HC S Sapphire, Helicone® HC S Scarabeus, Helicone® HC S Jade, and Helicone® HC S Maple by Wacker.

In another non-limiting embodiment of the present disclosure, the liquid crystal agent comprises cyclic polyorganosiloxanes grafted with cholesteric and biphenyl groups. Non-limiting examples of these cyclic polyorganosiloxanes are described by H. J. Eberle, A. Miller, F. H. Kreuzer in Liquid Crystals, 1989, Vol. 5, No.3, 907-916; and by J. Pinsl, Chr. Braüchie, F. H. Kreuzer in Journal of Molecular Electronics, Vol. 39-13 (1987); as well as in U.S. Pat. No. 4,410,570.

In a further non-limiting embodiment of the present disclosure, the liquid crystal agent is chosen from cyclomethicones grafted with cholesteric and biphenyl groups, as disclosed in patent application EP815826.

Non-limiting examples of liquid crystal agents conforming to this definition include the CL pigments sold by Wacker under the names SLM 41101 (Blue/Green), SLM 41102 (Red/Gold) and SLM 41103 (Yellow/Green).

The above liquid crystal agents may be employed alone, and/or coated onto inert supports such as micas, and/or in combination with other, non-liquid crystal agents.

In order to modify the glint obtained on the hair it is possible, for example, to apply, in addition to the liquid crystal agent, a colorant to the hair.

As used herein, the term “colorant” refers to soluble, non-liquid crystal dyes and/or non-liquid crystal pigments that are conventional and are suitable for coloring hair.

Non-limiting examples of these colorants include pigments resulting from the oxidizing polymerization of an indole derivative, as described in patent application FR 2 679 771. Additional examples of colorants include, but are not limited to organic or inorganic pigments that do not result from the oxidizing polymerization of indole compounds and which are cosmetically or dermatologically acceptable. These may be in the form of a pigmentary paste or powder.

Non-limiting examples of inorganic pigments include titanium dioxide (rutile or anatase), with or without surface treatment, which is codified in the Color Index under reference CI77891; black, yellow, red and brown iron oxides, codified under references CI77499, 77492 and 77491; manganese violet (CI77742); ultramarine blue (CI77007); hydrated chromium oxide (CI77289); and Prussian blue (CI77510).

Examples of organic pigments include, but are not limited to, Pigment Yellow 3, sold in particular under the trade name “Covanor Yellow W 1603” by Wacker (CI17710), D & C Red No. 19 (CI45170), D & C Red No. 9 (CI15585), D & C Red No. 21 (CI45380), D & C Orange No. 4 (CI5510), D & C Orange No. 5 (CI5370), D & C Red No. 27 (CI45410), D & C Red No. 3 (CI15630), D &C Red No. 7 (CI15850-I), D & C Red No. 6 (CI15850-2), D & C Yellow No. 5 (CI19140), D & C Red No. 36 (CI12085), D & C Orange No. 10 (CI45425), D & C Yellow No. 6 (CI15985), D & C Red No. 30 (CI73360), D & C Red No. 3 (CI45430), carbon black (CI77266), and lakes based on cochineal carmine (CI75470).

Nacreous pigments may also be used in the composition of the present disclosure. Non-limiting examples of nacreous pigments include white nacreous pigments such as mica coated with titanium oxide, bismuth oxide, colored nacreous pigments such as titanium mica with iron oxides, titanium mica with Prussian blue or with chromium oxide, and titanium mica with an organic pigment of precipitated type, and also those pigments based on bismuth oxichloride.

Further, organic pigment pastes may be used in the composition of the present disclosure. Non-limiting examples of these oprganic pigment pastes include the products sold by Hoechst under the following names:

COSMENYL YELLOW 10G: Pigment Yellow 3 (CI 11710)

COSMENYL YELLOW G: Pigment Yellow 1 (CI 11680)

COSMENYL ORANGE GR: Pigment Orange 43 (CI 71105)

COSMENYL RED R: Pigment Red 4 (CI 12085)

COSMENYL CARMINE FB: Pigment Red 5 (CI 12490)

COSMENYL VIOLET RL: Pigment Violet 23 (CI 51319)

COSMENYL BLUE A2R: Pigment Blue 15.1 (CI 74160)

COSMENYL GREEN GG: Pigment Green 7 (CI 74260)

COSMENYL BLACK R: Pigment Black 7 (CI 77266)

In addition, soluble dyes may be used in the composition of the present disclosure. Examples of these soluble dyes include, but are not limited to dyes belonging to the classes of the nitrobenzenes or heterocyclic dyes, quinonoid dyes (anthraquinones, naphthoquinones, benzoquinones), and azo dyes.

The composition of the present disclosure may contain the colorant in an amount ranging from 0.05-40%, such as 0.1-35%, for example, 0.25-25% by weight, relative to the total weight of the composition.

In a non-limiting embodiment of the present disclosure, when the composition comprises colorants, the liquid crystal agents to colorant weight ratio may range from 1/20 to 20/1, for example, from 1/10 to 10/1, such as from 1/5 to 5/1.

The present disclosure also relates to a method for treating keratin materials, such as hair, with the aforementioned composition. In a non-limiting embodiment of the present disclosure, this method includes applying the above-described composition to the keratin materials. In a further non-limiting embodiment of the present disclosure, the composition is applied to the keratin materials in the presence of a nucleophile. The method may be performed with or without heating.

In a non-limiting embodiment, the nucleophile is water, which may or may not be applied to the keratin material prior to the application of the composition.

In a non-limiting embodiment of the present disclosure, in order to modify the reaction kinetics, the keratin materials may be wetted beforehand with an aqueous solution whose pH has been adjusted by means of a base, an acid or an acid/base mixture. The acid and/or the base may be organic or inorganic. The aqueous solution may also be applied to the keratin material after the composition is applied.

The anionic polymerization kinetics may also be modified by pre-impregnating the keratin materials with a nucleophile. The nucleophile may be used in pure form, in solution form, as an emulsion, or may be encapsulated.

The nucleophiles capable of initiating the anionic polymerization are systems known per se which are capable of generating a carbanion on contact with a nucleophile, such as the hydroxyl ions present in water. As used herein, the term, “carbanion” refers to the chemical species defined in “Advanced Organic Chemistry”, Third Edition, by Jerry March, page 141.

The nucleophiles may be composed of a molecular compound, an oligomer, a dendrimer or a polymer possessing nucleophilic functions. Non-limiting examples of nucleophilic functions include: R₂N⁻, NH₂⁻, Ph₃C^{-, R}₃C⁻, PhNH⁻, pyridine, ArS⁻, R—C≡C⁻, RS⁻, SH, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph represents a phenyl group, Ar represents an aryl group and R represents a C₁-C₁₀ alkyl group.

In a non-limiting embodiment, the nucleophile is water, which may be applied to the keratin material prior to the application of the composition.

It another non-limiting embodiment, it is possible to modify the reaction kinetics by wetting the keratin material beforehand with an aqueous solution whose pH has been adjusted by a base, an acid or an acid/base mixture. The acid and/or the base may be organic or inorganic.

It is also possible to modify the anionic polymerization kinetics by pre-impregnating the keratin material with a nucleophile other than water. The nucleophile may be used in pure for, solution form, in the form of an emulsion, and may be encapsulated.

It is also possible to modify the anionic polymerization kinetics to enhance the nucleophilicity of the fiber by chemically converting the keratin material.

In a non-limiting embodiment of the present disclosure, the disulphide bridges, of which a keratin material is partly composed, may be reduced to thiols prior to application of the composition of the invention. Non-limiting examples of reducing agents that may be used for this purpose include, as reductants of the disulphide bridges of which the keratin is partly composed, the following:

anhydrous sodium thiosulphate

powdered sodium metabisulphite,

thiourea,

ammonium sulphite,

thioglycolic acid,

thiolactic acid,

ammonium thiolactate,

glyceryl monothioglycolate,

ammonium thioglycolate,

thioglycerol,

2,5-dihydroxybenzoic acid,

diammonium dithioglycolate,

strontium thioglycolate,

calcium thioglycolate,

zinc formaldehyde-sulphoxylate,

isooctyl thioglycolate,

dl-cysteine, and

monoethanolamine thioglycolate.

Further, it is possible to enhance the viscosity of the composition in order to alter the polymerization kinetics of the reaction. For example, the viscosity of the composition may be enhanced so as to reduce the polymerization rate of the monomers of the present disclosure. To accomplish this, it is possible to add one or more polymers which exhibit no reactivity with the monomers disclosed herein. Non-limiting examples of polymers that exhibit no reactivity with the monomers of the present disclosure include, poly(methyl methacrylate) (PMMA), and the cyanoacrylate-based copolymers that are described in U.S. Pat. No. 6,224,622.

In order to improve the adhesion of the poly(cyanoacrylate) formed in situ, the keratin material may be pretreated with a polymer, or may be subject to a hair treatment, such as direct dyeing, oxidation dyeing, permanent waving, or straightening, prior to the application of the composition of the present disclosure.

Application of the compositions of the present disclosure may or may not be followed by rinsing. Further these compositions may be in a variety of forms, such as a lotion, a spray, or a mousse.

In addition, the compositions of the present disclosure may be applied in a single step or in successive steps.

As a non-limiting example of the present disclosure, the method may comprise (a) applying to a keratin material at least one liquid crystal agent, and (b) applying to the keratin material at least one electrophilic monomer, in any order.

In a further non-limiting embodiment of the present disclosure, the one or more liquid crystal agents are applied to the keratin material before the one or more electrophilic monomers are applied.

As a non-limiting embodiment of the present disclosure, if the method of includes a number of steps, those steps may include:

a) applying aqueous solution to the hair, wherein the aqueous solution contains the liquid crystal agent of the present disclosure and, optionally, the non-liquid crystal colorant of the present disclosure, in an amount ranging from 0.05% to 40%, such as from 0.1% to 35%, for example, from 0.25% to 25% by weight;

b) applying a solution containing the electrophilic monomer of the present disclosure to the wetted hair, wherein the electrophilic monomer of the present disclosure is present in the solution in an amount ranging from 0.05% to 30%, such as from 0.01% to 50%, for example, from 0.1% to 20% by weight.

In addition solutions a) and b) may further contain conventional cosmetic additives. Further, the order of the two steps may be reversed. The first step may be preceded by the application of a cosmetic product, such as a care product. In addition, the last step may be succeeded by the application of a cosmetic product. Each step may be interrupted by rinsing and/or drying. Drying may be carried out under a hood, using a hairdryer and/or using hair straightener.

In another embodiment of the present disclosure, it is possible to perform multiple superpositions of layers using the above methods in order to attain a deposit having a desired chemical nature, mechanical strength, thickness, appearance, feel, etc.

In a further non-limiting embodiment of the present disclosure, the monomers are chosen from monomers that are capable of undergoing polymerization while present on the keratin fibers and under cosmetically acceptable conditions. The polymerization of the monomer may be accomplished at a temperature less than or equal to 80° C., such as from 10 to 80° C., for example, from 20 to 80° C., thereby allowing the application of the composition to be finished by drying under a hood, with blow drying or with passage of a straightening iron or curling tongs.

In another non-limiting embodiment, the present disclosure further relates to a kit comprising a first composition containing at least one electrophilic monomer, optionally at least one free-radical and/or anionic polymerization inhibitor, and a second composition comprising at least one liquid crystal agent in a cosmetically acceptable medium.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The following examples are intended to illustrate the invention without limiting the scope as a result.

### EXAMPLES

Tests were carried out using the following compounds:

monomer capable of undergoing anionic polymerization in the presence of a nucleophile: n-octyl 2-cyanoacrylate stabilized with 1% phosphoric acid:

liquid crystal colorant 1: Pigment CL SLM 41102, sold by Wacker

liquid crystal colorant 2: Pigment Helicone HC Scarabeus sold by Wacker

cosmetically acceptable medium:

50% of a mixture of polydimethylsiloxane alpha-omega dihydroxyl/cyclopentadimethylsiloxane (14.7/85.3) sold by Dow Corning under the name DC 1501 Fluid, and

50% of cyclopentadimethylsiloxane sold by Dow Corning under the name DC 245 Fluid.

Work was carried out with a 1 g tress of natural hair with a tone height of 4, which corresponds to a natural brown shade according to the classification of natural shades as described in “Science des Traitements Capillaires” by C. Zviak, Ed. Masson 1988, p. 278.

### Example 1

An aqueous solution was prepared with 10% of liquid crystal agent 1. 0.5 g of this aqueous solution was applied to a tress of clean, dry hair. The tress was subsequently dried under a hood. The tress was wetted with 0.5 g of water. 0.5 g of a solution of the cosmetic medium containing 10% of monomer was subsequently applied to the tress. After 10 minutes' exposure the tress was dried for 2 minutes with a hairdryer. The resulting tress possessed an attractive iridescent tint which varied from bronze to green depending on the angle of observation. The effect observed withstood at least six shampooings.

The mode of application may also be as follows: A solution in the cosmetic medium was prepared with 10% of liquid crystal colorant 1. The monomer was added so as to give a final monomer concentration of 10%. 0.5 g of this solution was applied to the tress, which has been wetted with 0.5 g of water. After 10 minutes of exposure, the tress was dried for 2 minutes with a hairdryer.

### Example 2

An aqueous solution was prepared with 10% of liquid crystal agent 2. 0.5 g of this aqueous solution was applied to a tress of clean, dry hair. The tress was subsequently dried under a hood. The tress was wetted with 0.5 g of water. 0.5 g of a solution of the cosmetic medium containing 10% of monomer was subsequently applied to the tress. After 10 minutes of exposure the tress was dried for 2 minutes with a hairdryer.

The resulting tress possessed an attractive tint that varied from blue to green depending on the angle of observation. The effect observed withstood at least six shampooings.

The mode of application may also be as follows:

A solution in the cosmetic medium was prepared with 10% of liquid crystal colorant 2. The monomer was added so as to give a final monomer concentration of 10%. 0.5 g of this solution was applied to the tress, which was pre-wetted with 0.5 g of water. After 10 minutes of exposure, the tress was dried for 2 minutes with a hairdryer.

### Example 3

### Methylheptyl Cyanoacrylate Monomer

The following composition was produced:

| | |
|---|---|
| DC 1501 Fluid | 40 g |
| DC 245 Fluid | 40 g |
| Helicone HC Scarabeus | 10 g |
| Methylheptyl cyanoacrylate from Chemence | 10 g |

1 g of the composition was applied to a 1 g tress of clean, wet hair. After a 15-minute interval, the tress was dried with a hairdryer for 2 minutes. The coloring of the resulting tress varied from violet to green depending on the angle of observation.

### Example 4

### Methylheptyl Cyanoacrylate Monomer with Acetic Acid

The following composition was produced:

| | |
|---|---|
| DC 1501 Fluid | 40 g |
| DC 245 Fluid | 39.75 g |
| Helicone HC Scarabeus | 10 g |
| Methylheptyl cyanoacrylate from Chemence | 10 g |
| Acetic acid | 0.25 g |

1 g of the composition was applied to a 1 g tress of clean, wet hair. After a 15-minute interval, the tress was dried with a hairdryer for 2 minutes. The coloring of the resulting tress varied from violet to green depending on the angle of observation.

### Example 5

### Methylheptyl Cyanoacrylate Monomer

The following composition was produced:

| | |
|---|---|
| DC 1501 Fluid | 40 g |
| DC 245 Fluid | 40 g |
| Helicone HC Scarabeus | 10 g |
| Ethylhexyl cyanoacrylate from Tong Shen | 10 g |

1 g of the composition was applied to a 1 g tress of clean, wet hair. After a 15-minute interval, the tress was dried with a hairdryer for 2 minutes. The coloring of the resulting tress varied from violet to green depending on the angle of observation.

### Example 6

### Butyl Cyanoacrylate Monomer

The following composition was produced:

| | |
|---|---|
| DC 1501 Fluid | 40 g |
| DC 245 Fluid | 39 g |
| Helicone HC Scarabeus | 10 g |
| Butyl cyanoacrylate B-60 from Tong Shen | 10 g |
| Acetic acid | 1 g |

1.5 g of the composition was applied to a 1 g tress of clean, wet hair. After a 15-minute interval, the tress was dried with a hairdryer for 2 minutes. The coloring of the resulting tress varied from violet to green depending on the angle of observation.

### Example 7

### Mixture of Methylheptyl Cyanoacrylate and Ethylhexyl Cyanoacrylate Monomers

The following composition was produced:

| | |
|---|---|
| DC 1501 Fluid | 40 g |
| DC 245 Fluid | 40 g |
| Helicone HC Scarabeus | 10 g |
| Methyheptyl cyanoacrylate from Chemence | 9 g |
| Ethylhexyl cyanoacrylate O-60 from Tong Shen | 1 g |

1 g of the composition was applied to a 1 g tress of clean, wet hair. After a 15-minute interval, the tress was dried with a hairdryer for 2 minutes. The coloring of the resulting tress varied from violet to green depending on the angle of observation.

### Example 8

### Mixture of Methylheptyl Cyanoacrylate and Butyl Cyanoacrylate Monomers

The following composition was produced:

| | |
|---|---|
| DC 1501 Fluid | 40 g |
| DC 245 Fluid | 40 g |
| Helicone HC Scarabeus | 10 g |
| Butyl cyanoacrylate B-60 from Tong Shen | 3 g |

1 g of the composition was applied to a 1 g tress of clean, wet hair. After a 15-minute interval, the tress was dried with a hairdryer for 2 minutes. The coloring of the resulting tress varied from violet to green depending on the angle of observation.

### Example 9

### Ethoxyethyl Cyanoacrlate Monomer

The following composition was produced:

| | |
|---|---|
| DC 1501 Fluid | 40 g |
| DC 245 Fluid | 35 g |
| Helicone HC Scarabeus | 10 g |
| Ethoxyethyl cyanoacrylate EO-460 from Tong Shen | 10 g |
| Acetic acid | 5 g |

1.5 g of the composition was applied to a 1 g tress of clean, wet hair. After a 15-minute interval, the tress was dried with a hairdryer for 2 minutes. The coloring of the resulting tress varied from violet to green depending on the angle of observation.

What is claimed is:

1. A composition for treating keratin materials, comprising, in a cosmetically acceptable medium, at least one electrophilic monomer and at least one liquid crystal agent.

2. The composition of claim 1, wherein said electrophilic monomer is chosen from monomers of formula (I): 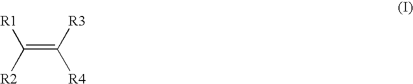
wherein:
R₁ and R₂, which may be identical or different, are chosen from:
a hydrogen atom;
saturated, unsaturated, linear, branched and cyclic hydrocarbon groups, containing 1 to 20 carbon atoms, and optionally containing one or more nitrogen, oxygen and/or sulphur atoms, which is optionally substituted by one or more groups chosen from —OR, —COOR, —COR, —SH, —SR, —OH and halogen atoms;
modified and non-modified polyorganosiloxane residues; and
polyoxyalkylene groups;
R₃ and R₄, which may be identical or different, are chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, —OH, linear and branched alkenyl groups, linear and branched alkynyl groups, C₁-C₄ mono- and polyfluoroalkyl groups, aryl groups, and aryloxy groups;
and R is chosen from saturated, unsaturated linear, branched and cyclic hydro-carbon groups containing 1 to 20 carbon atoms, and optionally containing one or more nitrogen, oxygen and/or sulphur atoms, which is optionally substituted by one or more groups chosen from —OR′, —COOR′, —COR′, —SH, —SR′, —OH and halogen atoms, and a polymer residue, R′denoting a C₁-C₁₀alkyl radical.

3. The composition of claim 2, wherein said electrophilic monomer is chosen from compounds of formula (II): 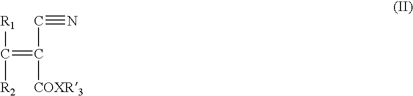
wherein:
X is chosen from NH, S and O, and
R′₃ denotes a hydrogen atom or a radical R.

4. The composition of claim 3, wherein said electrophilic monomer is chosen from C₁-C₂₀ polyfluoroalkyl 2-cyanoacrylates, (C₁-C₁₀ alkyl) cyanoacrylates, and (C₁-C₄ alkoxy)(C₁-C₁₀ alkyl) cyanoacrylates.

5. The composition of claim 4, wherein said electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

6. The composition of claim 1, wherein said electrophilic monomer is chosen from monomers of formula (III): 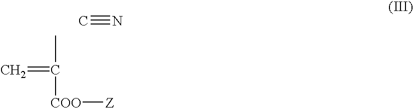
wherein,
Z is chosen from (CH₂)₇—CH₃, CH(CH₃)—(CH₂)₅—CH₃, CH₂—CH(C₂H₅)—(CH₂)₃—CH₃, (CH₂)₅—CH(CH₃)—CH₃, and (CH₂)₄—CH(C₂H₅)—CH₃.

7. The composition of claim 1, wherein said electrophilic monomer is present in the composition in an amount ranging from about 0.001% to about 80% by weight, relative to the total weight of the composition.

8. The composition of claim 7, wherein said electrophilic monomer is present in the composition in an amount ranging from about 1% to about 20% by weight, relative to the total weight of the composition.

9. The composition of claim 1, wherein said electrophilic monomer is attached to a support via one or more covalent bonds.

10. The composition of claim 9, wherein said support is chosen from polymers, oligomers and dendrimers.

11. The composition of claim 1, wherein said cosmetically acceptable medium is anhydrous.

12. The composition of claim 11, wherein said cosmetically acceptable medium is chosen from organic oils, silicones, mineral oils, vegetable oils, waxes, C₅-C₁₀ alkanes, acetone, methyl ethyl ketone, esters of C₁-C₂₀ acids and C₁-C₈ alcohols, dimethoxyethane, diethoxyethane, C₁₀-C₃₀ fatty alcohols, C₁₀-C₃₀ fatty acids, C₁₀-C₃₀ fatty amides, C₁₀-C₃₀ fatty alcohol esters, and mixtures thereof.

13. The composition of claim 1, wherein said composition further comprises one or more polymerization inhibitors.

14. The composition of claim 13, wherein said polymerization inhibitors are chosen from free-radical, and anionic polymerization inhibitors.

15. The composition of claim 14, wherein said polymerization inhibitors are chosen from sulphur dioxide, nitric oxide, lactone, boron trifluoride, hydroquinone and its derivatives, benzoquinone and its derivatives, catechol and its derivatives, anisole and its derivatives, pyrogallol, 2,4-dinitrophenol, 2,4,6-trihydroxybenzene, p-methoxyphenol, hydroxybutyltoluene, alkyl sulphates, alkyl sulphites, alkyl sulphones, alkyl sulphoxides, alkyl sulphides, mercaptans, 3-sulpholene, and mixtures thereof.

16. The composition of claim 13, wherein said polymerization inhibitors are present in the composition in an amount ranging from 10 ppm to 20% by weight, relative to the total weight of the composition.

17. The composition of claim 16, wherein said polymerization inhibitors are present in the composition in an amount ranging from 10 ppm to 1% by weight, relative to the total weight of the composition.

18. The composition of claim 1, wherein said liquid crystal agent is chosen from:
compounds containing a cholesteric function of the structure: 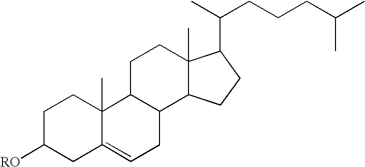
wherein:
R comprises a branched or unbranched alkyl or alkylcarbonyl group containing 1 to 30 carbon atoms which is unsubstituted or substituted by cyclic, aromatic and/or halogen groups;
compounds of the formula:
Aₗ^{5′}-[(X¹)ₐ-(A¹)_{b}-(A²)_{c}]_{d}-Zₑ-[(X²)_{f}-(A³)_{g}-(A⁴)ₕ]ᵢ-Aₖ⁵
wherein,
X¹ and X², which may be the same or different, are chosen from divalent —O—, —COO—, —CONH—, —CO—, —S—, —C≡C—, —CH═CH—, —CH₂—CH₂, —, —CH═N—, —N═N— or —N═N(O)—, —CH═N—N═CH—, —CH═CH—COO—, and —OCO—CH═CH— radicals,
A¹, A², A³ and A⁴, which may be the same or different, are chosen from divalent 1,4-phenylene, 1,4-cyclohexylene, and optionally substituted arylene, heteroarylene, heterocycloalkylene and cycloalkylene radicals,
Z is chosen from divalent, trivalent to tetravalent, benzene-1,4-cyclohexane and benzene-1,3-cyclopentane radicals,
A⁵ and A^{5′}, which may be the same or different, are chosen from saturated and unsaturated alkyl, alkoxy and cycloalkyl radicals having 1 to 16 carbon atoms, a steroidal radical, a halogen, a hydrogen atom, hydroxyl, nitrile, and trialkylsilyloxy radicals,
a, b, c, d, f, g, h, i, k, and l, which may be the same or different, denote an integer ranging from 0 to 3,
e is 0 or 1,
the sum of a+b+c+d+e+f+g+h+i+k is greater than or equal to 2,
the sum of d+i is less than or equal to 4, and
with the proviso that this group does not include a peroxide radical;
a polymer obtained by the polymerization of mesogenic monomers having the following structure:
Y¹—B¹-M-R
wherein,
Y¹ is a polymerizable group that is chosen from acrylate, methacrylate, epoxy, isocyanate, hydroxyl, vinyl ether, vinyl ester, styryl, and trialkoxysiloxy groups,
B¹ is a —CₙH₂ₙ— group, in which n is an integer ranging from 0 to 20 and one or more methylene groups of said —CₙH₂ₙ— group may be substituted by a halogen atom or, when said methylenes are not adjacent, may be replaced by one or more groups chosen from —O—, —NH—, —OCO—, —OCO—O—, —S—CO—COO—, —CONH—, —CO—, —S—, —C≡C—, and —CH═CH—,
M represents a group of formula:
—[(X¹)ₐ-(A¹)_{b}-(A²)_{c}]_{d}-Zₑ-[(X²)_{f}-(A³)_{g}-(A⁴)ₕ]ᵢ-
in which:
X¹ and X², which may be the same or different, are chosen from divalent —O—, —COO—, —CONH—, —CO—, —S—, —C≡C—, —CH═CH—, —CH₂—CH₂—, —CH═N—, —N═N— or —N═N(O)—, —CH═N—N═CH—, —CH═CH—COO— and —OCO—CH═CH— radicals,
A¹, A², A³ and A⁴, which may be the same or different, are chosen from divalent 1,4-phenylene, 1,4-cyclohexylene, optionally substituted arylene, heteroarylene, cycloalkylene, and heterocycloalkylene radicals,
Z is chosen from divalent, trivalent to tetratvalent benzene-1,4-cyclohexane and benzene-1,3-cyclopentane radicals, and divalent chiral groups that contain at least 4 carbon atoms, wherein the divalent chiral group includes at least one asymmetric carbon.

19. The composition of claim 18, wherein said liquid crystal agent is chosen from cholesterol erucyl carbonate, cholesterol methyl carbonate, cholesterol oleyl carbonate, cholesterol para-nonylphenyl carbonate, cholesterol phenyl carbonate, cholesterol acetate, cholesterol benzoate, cholesterol butyrate, cholesterol isobutyrate, cholesterol chloride, cholesterol chloroacetate, cholesterol cinnamate, cholesterol crotanoate, cholesterol decanoate, cholesterol erucate, cholesterol heptanoate, cholesterol hexanoate, cholesterol myristate, cholesterol nonanoate, cholesterol octanoate, cholesterol oleate, cholesterol propionate, cholesterol valerate and dicholesteryl carbonate.

20. The composition of claim 18, wherein said liquid crystal agent is Polyacrylate-4.

21. The composition of claim 1, wherein said liquid crystal agent is chosen from cyclic polyorganosiloxanes that are grafted with one or more cholesteric and/or biphenyl groups.

22. The composition of claim 1, wherein said liquid crystal agent is present in the composition in an amount ranging from 0.05% to 40% by weight, relative to the total weight of the composition.

23. The composition of claim 22, wherein said liquid crystal agent is present in the composition in an amount ranging from 0.25% to 25% by weight, relative to the total weight of the composition.

24. The composition of claim 1, further comprising at least one agent chosen from reducing agents, fats, plasticizers, softeners, antifoams, moisturizers, pigments, clays, mineral fillers, UV filters, mineral colloids, peptizers, solubilizers, perfumes, preservatives, anionic, cationic, nonionic or amphoteric surfactants, fixative or non-fixative polymers, polyols, proteins, vitamins, direct dyes or oxidation dyes, and pearlizers.

25. The composition of claim 1, wherein said composition is in the form of a lotion, spray or mousse.

26. A cosmetic composition, comprising, in a cosmetically acceptable medium, at least one electrophilic monomer and at least one liquid crystal agent other than cholesteric liquid crystal agents.

27. The composition of claim 26, wherein said electrophilic monomer is chosen from those of formula (I): 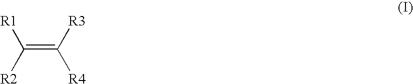
wherein,
R₁ and R₂, which may be identical or different, are chosen from:
a hydrogen atom;
saturated, unsaturated, linear, branched and cyclic hydrocarbon groups, containing 1 to 20 carbon atoms, and optionally containing one or more nitrogen, oxygen and/or sulphur atoms, which is optionally substituted by one or more groups chosen from —OR, —COOR, —COR, —SH, —SR, —OH and halogen atoms;
modified and non-modified polyorganosiloxane residues; and
polyoxyalkylene groups;
R₃ and R₄, which may be identical or different, are chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, —OH, linear and branched alkenyl groups, linear and branched alkynyl groups, C₁-C₄ mono- and polyfluoroalkyl groups, aryl groups, and aryloxy groups;
and R is chosen from saturated, unsaturated linear, branched and cyclic hydro-carbon groups containing 1 to 20 carbon atoms, and optionally containing one or more nitrogen, oxygen and/or sulphur atoms, which is optionally substituted by one or more groups chosen from —OR∝, —COOR′, —COR′, —SH, —SR′, —OH and halogen atoms, and a polymer residue, R′ denoting a C₁-C₁₀ alkyl radical.

28. The composition of claim 26, wherein said electrophilic monomer is chosen from those of formula (II): 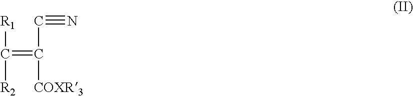
wherein,
X is chosen from NH, S and O; and
R′₃ is a hydrogen atom or a radical R.

29. The composition of claim 26, wherein said electrophilic monomer is chosen from C₁-C₂₀ polyfluoroalkyl 2-cyanoacrylates, (C₁-C₁₀ alkyl) cyanoacrylates, and (C₁-C₄ alkoxy)(C₁-C₁₀ alkyl) cyanoacrylates.

30. The composition of claim 26, wherein said electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

31. The composition of claim 26, wherein said electrophilic monomer is chosen from monomers of formula (III): 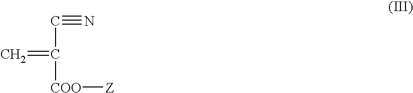
wherein,
Z is chosen from (CH₂)₇—CH₃, CH(CH₃)—(CH₂)₅—CH₃, CH₂—CH(C₂H₅)—(CH₂)₃—CH₃, (CH₂)₅—CH(CH₃)—CH₃, and (CH₂)₄—CH(C₂H₅)—CH₃.

32. The composition of claim 26, wherein the liquid crystal agent is chosen from:
compounds defined by the following formula:
Aₗ^{5′}-[(X¹)ₐ-(A¹)_{b}-(A²)_{c}]_{d}-Zₑ-[(X²)_{f}-(A³)_{g}-(A⁴)ₕ]ᵢ-Aₖ⁵
wherein,
X¹ and X², which may be the same or different, are chosen from divalent —O—, —COO—, —CONH—, —CO—, —S—, —C≡C—, —CH═CH—, —CH₂—CH₂,—, —CH═N—, —N═N— or —N═N(O)—, —CH═N—N═CH—, —CH═CH—COO—, and —OCO—CH═CH— radicals,
A¹, A², A³ and A⁴, which may be the same or different, are chosen from divalent 1,4-phenylene, 1,4-cyclohexylene, and optionally substituted arylene, heteroarylene, heterocycloalkylene and cycloalkylene radicals,
Z is chosen from divalent, trivalent totetravalent, benzene-1,4-cyclohexane and benzene-1,3-cyclopentane radicals,
A⁵ and A^{5′}, which may be the same or different, are chosen from saturated and unsaturated alkyl, alkoxy and cycloalkyl radicals having 1 to 16 carbon atoms, a steroidal radical, a halogen, a hydrogen atom, hydroxyl, nitrile and trialkylsilyloxy radicals,
a, b, c, d, f, g, h, I, k, and 1, which may be the same or different, denote an integer ranging from 0 to 3,
e is 0 or 1,
the sum of a+b+c+d+e+f+g+h+i+k is greater than or equal to 2,
the sum of d+i is less than or equal to 4, and
with the proviso that this group does not include a peroxide radical;
a polymer obtained by the polymerization of mesogenic monomers having the strucutre:
Y¹—B¹-M-R
wherein,
Y¹ is a polymerizable group that is chosen from acrylate, methacrylate, epoxy, isocyanate, hydroxyl, vinyl ether, vinyl ester, styryl, and trialkoxysiloxy groups,
B¹ is a —CₙH₂ₙ— group, in which n is an integer ranging from 0 to 20 and one or more methylene groups of said —CₙH₂ₙ— group may be substituted by a halogen atom or, when said methylenes are not adjacent, may be replaced by one or more groups chosen from —O—, —NH—, —OCO—, —OCO—O—, —S—CO—COO—, —CONH—, —CO—, —S—, —C≡C—, and —CH═CH—,
M represents a group of formula:
—[(X¹)ₐ-(A¹)_{b}-(A²)_{c}]_{d}-Zₑ-[(X²)_{f}-(A³)_{g}-(A⁴)ₕ]ᵢ-
in which:
X¹ and X², which may be the same or different, are chosen from divalent —O—, —COO—, —CONH—, —CO—, —S—, —C≡C—, —CH═CH—, —CH₂—CH₂,—, —CH═N—, —N═N— or —N═N(O)—, —CH═N—N═CH—, —CH═CH—COO— and —OCO—CH═CH— radicals,
A¹, A², A³ and A⁴, which may be the same or different, are chosen from divalent 1,4-phenylene, 1,4-cyclohexylene, optionally substituted arylene, heteroarylene, cycloalkylene, and heterocycloalkylene radicals,
Z is chosen from divalent, trivalent to tetratvalent benzene-1,4-cyclohexane, benzene-1,3-cyclopentane radicals, and divalent chiral groups that contain at least 4 carbon atoms, wherein the divalent chiral group includes at least one asymmetric carbon.

32. The composition of claim 31, wherein said liquid crystal agent is Polyacrylate-4.

33. The composition of claim 26, wherein said liquid crystal agent is chosen from cyclic polyorganosiloxanes that are grafted with one or more cholesteric and/or biphenyl groups.

34. A method for treating keratin materials, comprising:
applying at least one liquid crystal agent to said keratin material; and
applying at least one electrophilic monomer to said keratin material.

35. The method of claim 34, wherein the liquid crystal agent is applied to the keratin material before the electrophilic monomer.

36. A method of treating keratin materials, comprising:
applying, in the presence of a nucleophile, a composition comprising a cosmetically acceptable medium to said keratin material, wherein said cosmetically acceptable medium comprises one or more electrophilic monomers and one or more liquid crystal agents.

37. The method of claim 36, wherein the nucleophile is chosen from molecular compounds, oligomers, dendrimers, and polymers, wherein said molecular compounds, oligomers, dendrimers and polymers comprise nucleophilic functionalities chosen from: R₂N⁻, NH₂⁻, Ph₃C⁻, R₃C⁻, PhNH⁻, pyridine, ArS—, R—C≡C⁻, RS⁻, SH⁻, RO—, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, where Ph represents a phenyl group, Ar represents an aryl group, and R represents a C₁-C₁₀ alkyl group.

38. The method of claim 37, wherein the nucleophile is water.

39. The method of claim 36, wherein said keratin material has been wetted with an aqueous solution whose pH has been adjusted by a base, an acid or an acid/base mixture prior to the application of said composition.

40. The method of claim 36, wherein said keratin material has been impregnated with a nucleophile other than water prior to the application of said composition.

41. The method of claim 36, wherein said keratin materials are reduced with a reducing agent prior to the application of said composition.

42. The method of claim 41, wherein said reducing agent is chosen from anhydrous sodium thiosulphate, powdered sodium metabisulphite, thiourea, ammonium sulphite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formaldehyde-sulphoxylate, isooctyl thioglycolate, dl-cysteine and monoethanolamine thioglycolate.

43. The method of claim 36, wherein said composition further comprises a polymer chosen from poly(methyl methacrylate) and cyanoacrylate-based copolymers.

44. The method of claim 36, further comprising a rinsing after the composition is applied to the keratin material.

45. A kit comprising a first composition containing at least one electrophilic monomer and optionally at least one free-radical and/or anionic polymerization inhibitor, and a second composition comprising a cosmetically acceptable medium, said cosmetically acceptable medium comprising at least one liquid crystal agent.
